(19) United States
(12) Patent Application Publication
(10) Pub. No.: US 2024/0188953 A1
(43) Pub. Date: Jun. 13, 2024

(54) Suture Ferrule Retaining System

(71) Applicant: Cypris Medical, Inc., Chicago, IL (US)

(72) Inventors: Daniel E. Roush, Niles, IL (US); Daniel B. Holton, Evanston, IL (US); Gregory D. Lyons, Carmel, IN (US)

(73) Assignee: Cypris Medical, Inc., Chicago, IL (US)

(21) Appl. No.: 18/079,615

(22) Filed: Dec. 12, 2022

(51) Int. Cl. A61B 17/04; A61B 17/06

(52) U.S. Cl. CPC A61B 17/0469; A61B 17/06; A61B 2017/0472

(57) ABSTRACT

A needle adapted to insert into a ferrule when the ferrule resides in a ferrule hollow formed with surface of a suturing device probe. The needle includes a first section and second section. When the needle is oriented in a suturing commenced state, stage 1a, the needle first section resides in a needle receiver hollow formed by the ferrule. The second section of the needle resides outside of the needle receiver hollow. When the needle is oriented in a suturing commend state, stage 1b, the first and second needle section both reside in the needle receiver hollow.

### I. FIELD OF THE INVENTION

A needle adapted to insert into a ferrule when said ferrule resides in a ferrule hollow formed with surface of a suturing device probe.

### II. BACKGROUND OF THE INVENTION

U.S. Pat. No. 10,631,855, Wound Closure Device Including Direct Driven Needle, Robert C Smith, concerns a suturing device which includes a housing, a rod, a ferrule assembly, and a needle. The housing defines a guide lumen extending longitudinally therethrough. The rod is engaged with the housing and extends distally therefrom. The ferrule assembly is disposed at a distal end of the rod and is configured to releasably retain a ferrule therein. The ferrule is adapted to retain of a portion of a suture therein. The needle is configured for translation through the guide lumen defined within the housing. A distal end of the needle is configured to engage the ferrule within the ferrule assembly for removal of the ferrule from the ferrule assembly.

U.S. Pat. No. 10,390,818, Ferrule for Use with a Minimally Invasive Suturing Device, Mark William Kyser, et al., concerns a ferrule for use with a surgical suturing device. The ferrule has a suture interface and a needle receptacle. The ferrule also has one or more indentations protruding into the needle receptacle. The ferrule further has one or more slits adjacent the one or more indentations. Another ferrule is disclosed for use with a surgical suturing device. The ferrule has a suture interface and a needle receptacle. The ferrule also has three indentations protruding into the needle receptacle. The ferrule further has three slits, each of the slits substantially centered between a different pair of indentations from the three indentations, and wherein each of the slits is longer and thinner than any one of the three indentations.

U.S. Pat. No. 10,231,729, Ferrule Holder with Suture Relief, Jud S, Sauer, concerns a surgical suturing instrument that has a chamber for receiving and aligning a ferrule with a reciprocating needle. The chamber includes a plurality of protuberant concave and convex surfaces for positioning and aligning the ferrule within the chamber and the plurality of suture receiving chambers disposed between the aligning ridges for receiving a suture and preventing the suture from jamming the ferrule in the chamber.

### III. SUMMARY OF THE INVENTION

An example of the present invention concerns a needle adapted to insert into a ferrule wherein said ferrule resides in a ferrule hollow formed with surface of a suturing device probe. The ferrule includes a suture retainer connected to a needle receive. The suture retainer connects a suture to the needle receiver. An internal surface of the needle receiver delimits a needle receiving hollow.

The needle includes a first end portion. A tip at the first end portion of said needle. The tip includes an external surface which tapers to a point. The needle further includes an engagement first section. The tip extends away form said needle engagement first section. An external surface of the engagement first section extends about a longitudinal axis of the engagement first section. The external surface delimits a first diameter.

The needle further includes an engagement second section having a second diameter. The second diameter is greater than said first diameter. An external surface of the engagement second section extends about a longitudinal axis of the second engagement second section. The external surface delimits a second diameter, an external surface delimiting an end portion, said external surface delimiting said end portion. The second end portion delimiting a configuration selected from a group of configurations consisting of a taper tapering in the direction of the tip, a shoulder, a corner, a portion outward of said engagement first section external surface, and combinations thereof. The engagement first section extends away from said engagement second section towards said tip.

When the needle is oriented in a suturing commenced state, stage 1a, the tip of the needle and the ferrule engagement first section of the needle reside in the needle receiving hollow. The internal surface of the needle receiver delimiting the hollow imparts a force on the ferrule engagement first section strong enough to hold the needle in the needle receiver during retraction of the needle from the ferrule hollow, towards a progression hollow exit formed by surface of the suturing device probe, and through a substrate in a chamber of the suturing device probe. An inner diameter of a circle delimited by the internal surface of the needle receiver too short to accommodate said ferrule engagement second section in said needle receiver hollow. The engagement second section resides outside the needle receiver.

When the needle is oriented in a suturing commenced state, stage 1b, the internal surface of the needle receiver delimiting the hollow imparts a force on the ferrule engagement second section strong enough to hold the needle in the needle receiver during retraction of the needle from the ferrule hollow of the suturing device probe, towards the progression hollow exit, and through the substrate in the chamber of the suturing device probe. The inner diameter of the circle delimited by the internal surface of the needle receiver too long to enable the internal surface of the needle receiver to impart the force on the engagement first section strong enough to hold the needle in the needle receiver during retraction of the needle from the ferrule hollow of the suturing device probe, towards said progression hollow, and through said substrate in said chamber of the suturing device probe.

### IV. BRIEF DESCRIPTION OF THE DRAWINGS

FIG. 1 shows an isometric view of a suturing device embodying features of the present invention.

FIG. 2 shows a plan view of the suturing device shown in FIG. 1.

FIG. 3A shows a closeup, bottom, isometric view of the probe of the suiting device shown FIG. 1.

FIG. 3B shows a closeup exploded view of the probe of the suturing device shown in FIG. 3A.

FIG. 4A shows a cross section of the probe of the suturing device shown in FIG. 1 taken along the view lines shown in FIG. 1; the ferrule, holder, retainer and needle resides in the probe.

FIG. 4B shows the cross section of the probe shown in FIG. 4A with the ferrule exploded from the probe and the holder and retainer exploded from the probe.

FIG. 5A shows a side view of the ferrule shown in FIG. 5B; the ferrule carries a suture.

FIG. 5B shows a cross section of the ferrule and suture shown in FIG. 5A.

FIG. 6A shows a across section like the cross section shown in FIG. 4A; the first section of the needle extends into the receiver of the ferrule; the second section of the needle remains outside the receiver; the elongated suture extends from the suture retainer of the ferrule.

FIG. 6B shows the cross section shown in FIG. 6A except the needle is partially retracted and the ferrule has been pulled out of a ferrule hollow of the probe.

FIG. 7A shows a cross section like the cross section shown in FIG. 4A, except it includes an elongated suture connected to the suture retainer of the ferrule.

FIG. 7B shows a cross section like the cross section shown in FIG. 7A; the first section and second section of the needle extends into the receiver of the ferrule; the elongated suture extends from the suture retainer of the ferrule.

FIG. 7C shows the cross section shown in FIG. 7B except the needle is partially retracted and the ferrule has been pulled out of the ferrule hollow of the probe.

FIG. 7D shows the cross section shown in FIG. 7C except the needle is fully retracted and the ferrule remains on the needle.

FIG. 8A shows a cross section like that shown in FIG. 7A except tissue resides in the substrate capture chamber

FIG. 8B shows a cross section like that shown in FIG. 8A except the needle has passed through the tissue and the needle's first and second sections reside the ferrule receiver

FIG. 8C shows a cross section like that shown in FIG. 8B except the needle has been partially retracted and the ferrule with the needle inserted resides in the tissue.

FIG. 8D shows a cross section like that shown in FIG. 8C except the needle has been fully retracted and the ferrule, with the needle inserted, has passed through the tissue and resides in the probe.

FIG. 9A shows a cross section like the cross section shown in FIG. 7C; except the needle has a first and second section blunter than the needle shown in FIG. 7A.

FIG. 9B shows the cross section of FIG. 9A; except the needle with the ferrule thereon resides retracted.

Before the invention is explained in detail, it is to be understood that the invention is not limited in its application to the details of construction and arrangement of components set forth in the following description or as illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings. Further the phrase “resides in” does not mean completely resides in. It encompasses partially resides in. The term hollow as used herein is broad enough to include void space that is delimited. Further the phrase “delimited” or “delimited by” includes partially delimited. Further, although the description uses a needle to describe a feature of the invention, the invention more broadly applies to a ferrule carrier an example of which is a needle. Further the description includes reference to a substrate. An example of a substrate is human tissue.

### V. DETAILED DESCRIPTION OF THE INVENTION

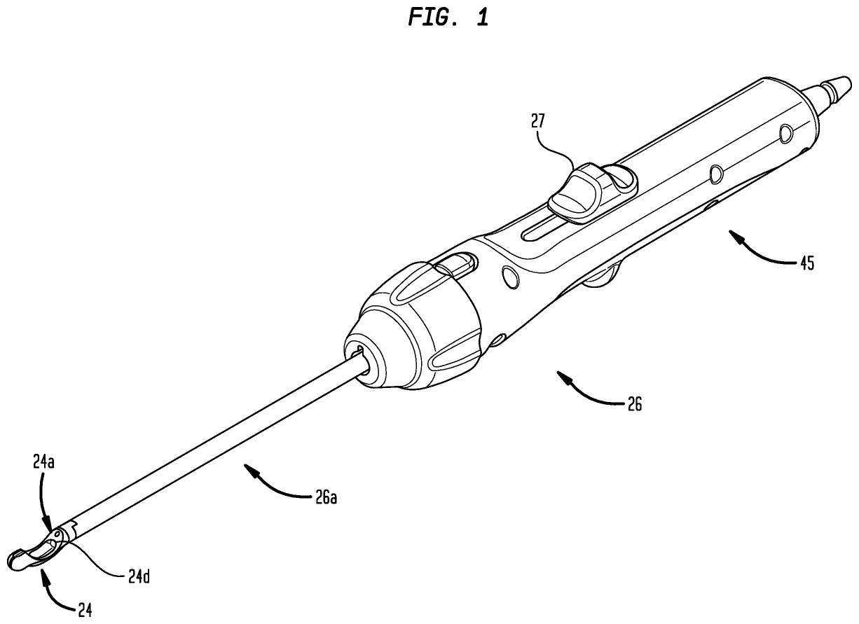

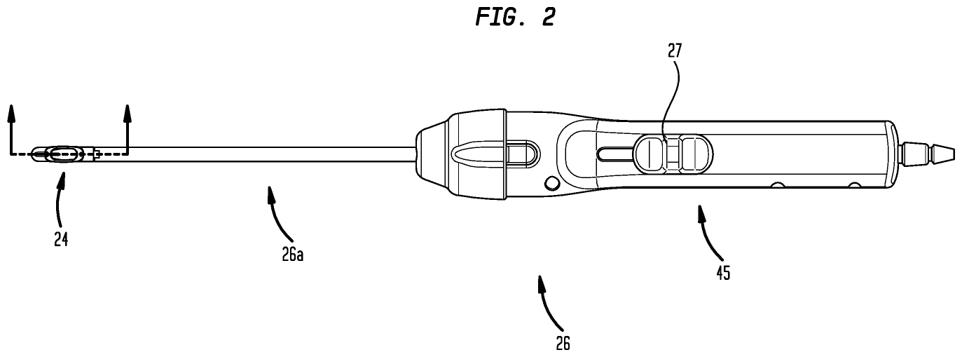

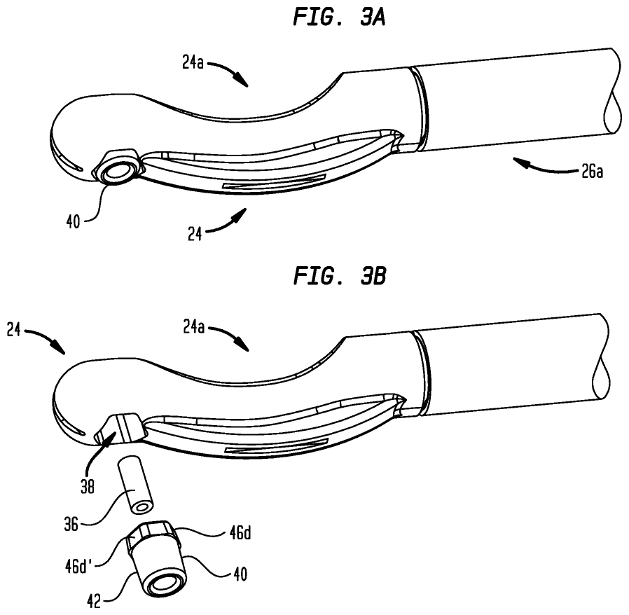

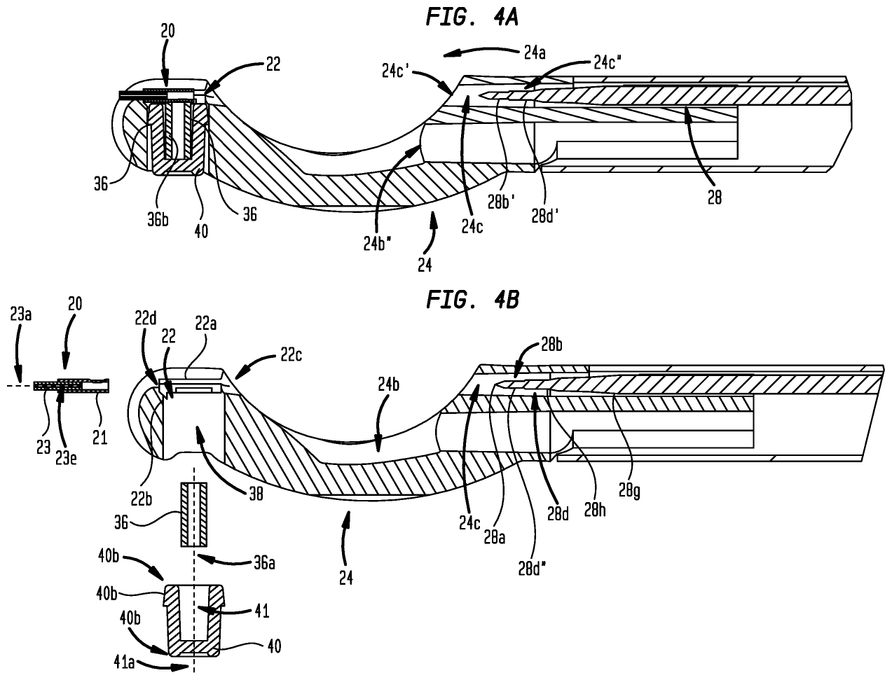

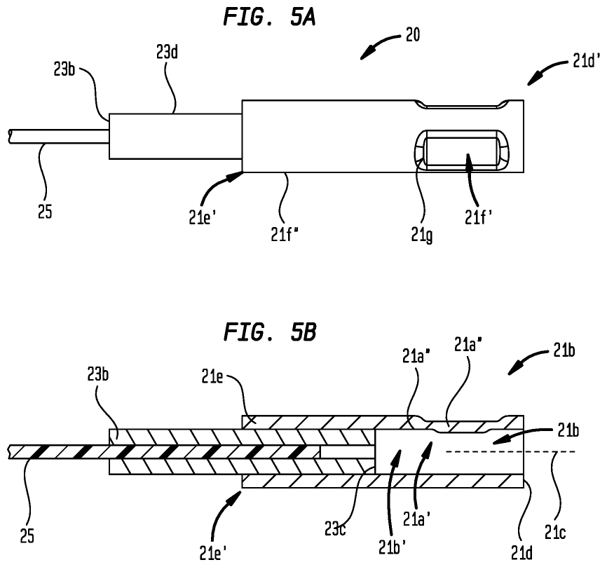

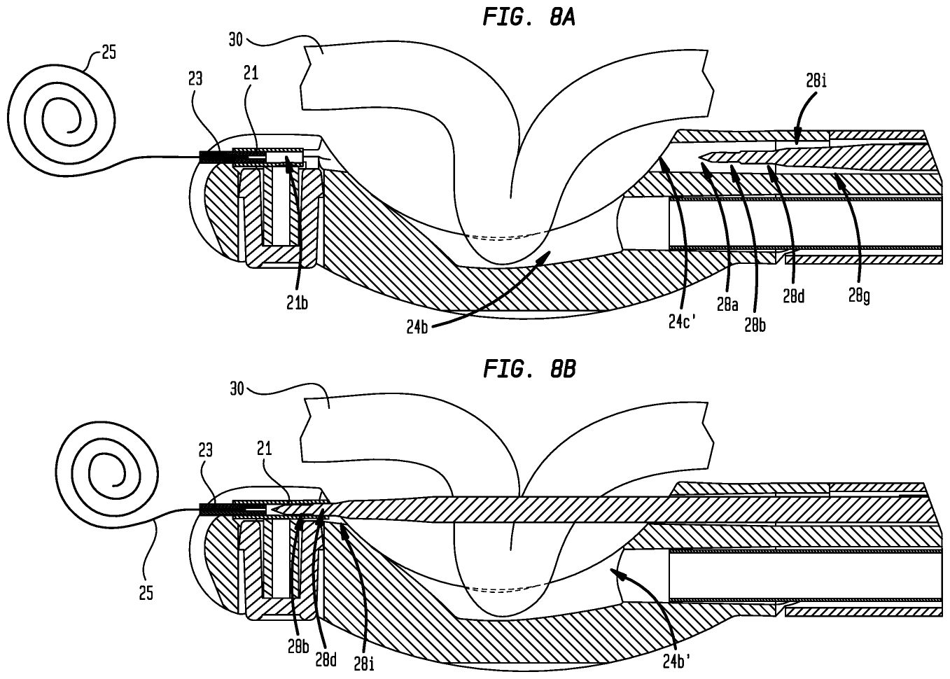

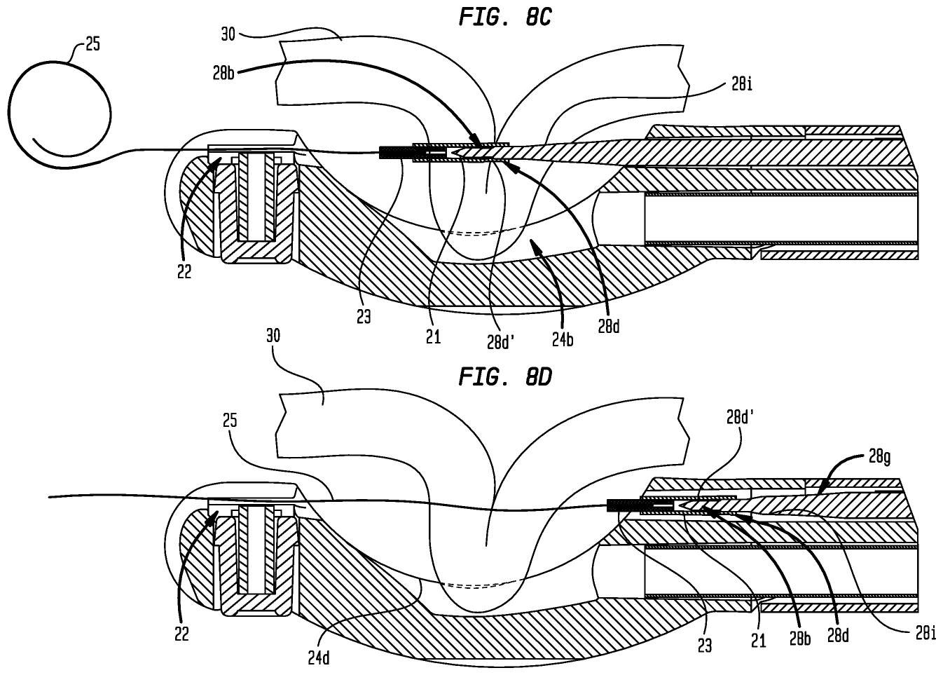

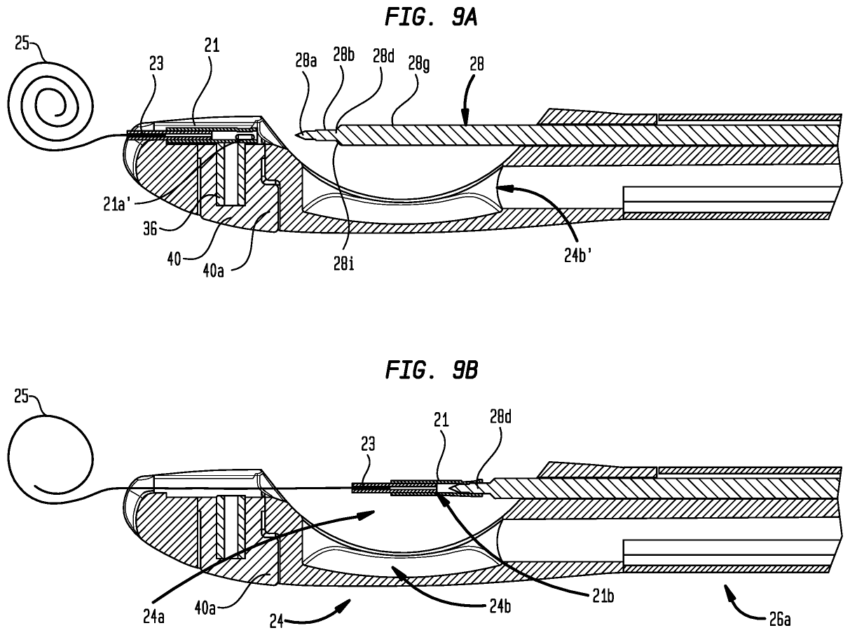

Now referring to FIGS. 1-9B, when a ferrule (20) resides in a suturing ready state, the ferrule (20) removably resides in a ferrule hollow (22) delimited by a surface (22a) of a suture device probe (24). The surface delimiting the ferrule hollow includes a recessed portion forming a recessed area (22b) in which seats a needle receiver (21) of the ferrule (20). The needle receiver (21) includes projections (21a′) which delimits a needle receiver hollow (21b) and which projects inward towards a needle receiver longitudinal axis (21c). The receiver hollow and projections delimited by internal surface (21a) of the needle receiver.

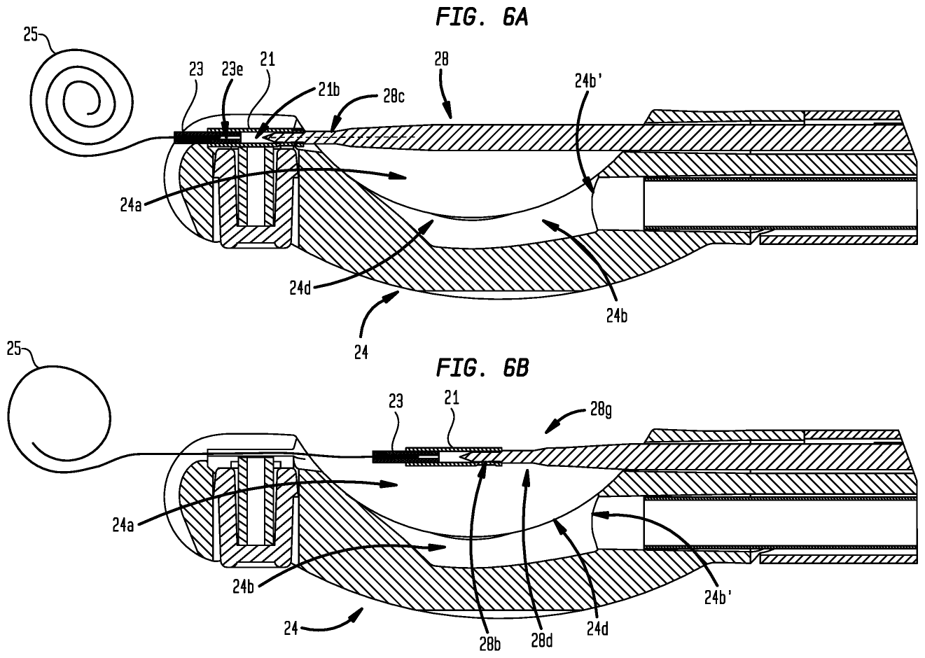

The suture device (26) carries a needle (28) which resides in a tube (26a) of the suture device. A tip (28a) and ferrule engagement first section (28b) of the needle extends into the needle receiver hollow (21b) when the needle (21) resides in the suturing commenced state, stage 1a. A Ferrule engagement second section (28d) remains outside the needle receiver hollow. See FIG. 6A. The ferrule engagement first section (28b) has an external surface (28b′) about a longitudinal axis (28c) of the ferrule engagement first section (28b). The external surface (28b′) delimits a diameter. The projections (21a′) impart a force on the ferrule engagement first section (28b) strong enough to hold the needle inside the needle receiver hollow (21b) during retraction of the needle (28). During retraction of the needle, the needle (28) retracts the ferrule (20) from the ferrule hollow (22) and through a substrate (30). The substrate (30) in a substrate capture chamber (24b) formed with a surface of the probe (24). The imparted force can exceed 5 newtons; it can exceed 10 newtons; it can exceed 19 newtons; it can exceed 30 newtons. It can be from 10 to 30 newtons; it can be from 15 to 30 newtons it can be from 15 to 25 newtons. It can be about 20 newtons+−3 newtons. A ferrule engagement second section (28d) resides in a position relative to said projections (21a′) of said needle receiver (21) delimiting said hollow (21b) wherein said projections (21a′) fail to impart a force on the engagement second section (28d) strong enough to hold the needle in the needle receiver during retraction of the needle from the ferrule hollow of the suturing device, towards a progression hollow exit (24c′) formed by surface of said probe, and through said substrate in said chamber of the suturing device probe. The inner diameter of a circle delimited and bounded by the projections (21a′) and a circle delimited and bounded by unprojected internal surface (21a″) are not large enough to accommodate the ferrule engagement second section (28d). The second section having a diameter delimited by its external surface (28d′), greater than the diameter of the engagement first section (28b).

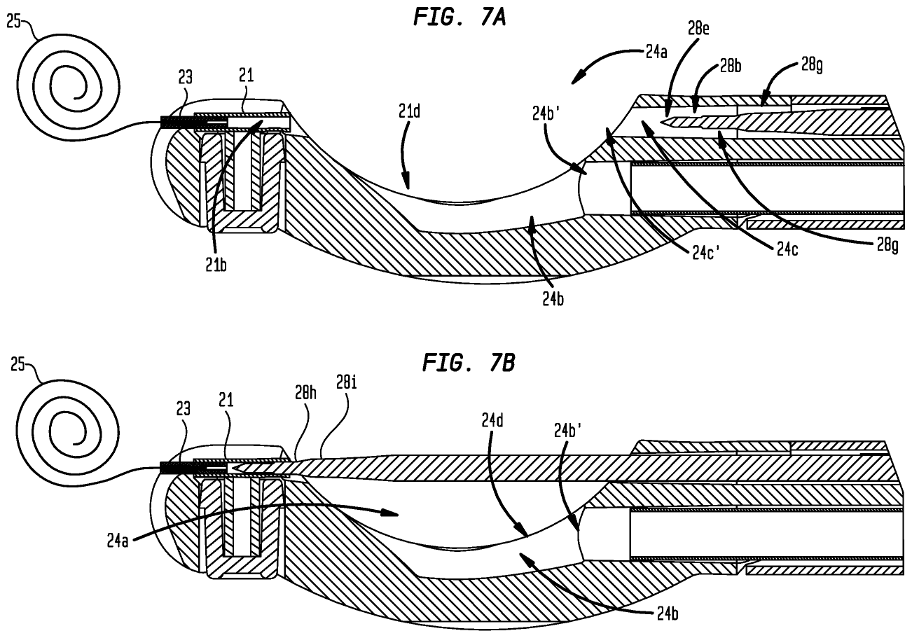

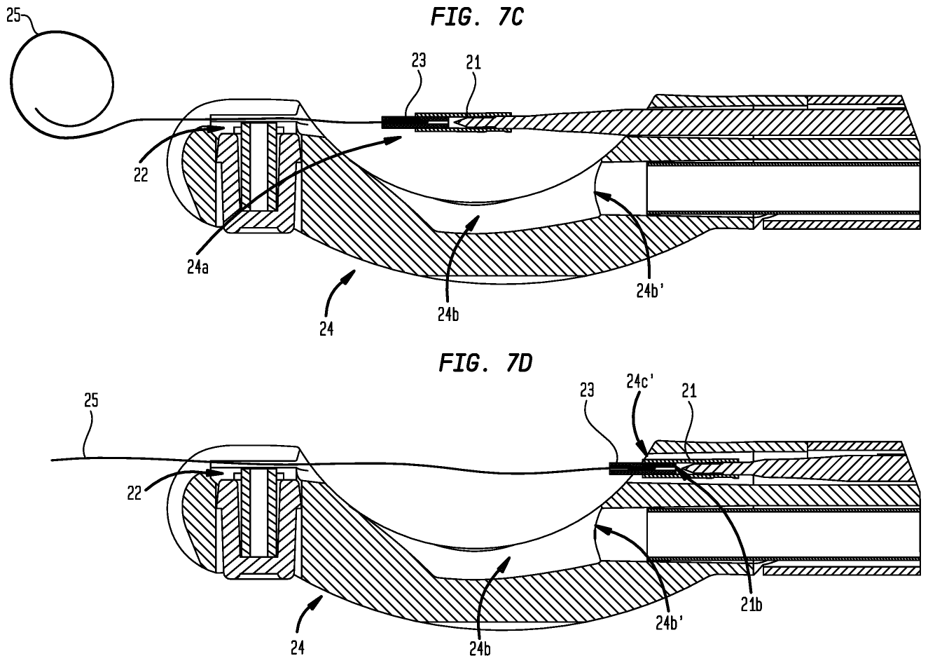

The ferrule engagement second section (28d) has an end portion (28d″). External surface delimiting said end portion (28d″) delimits a configuration selected from a group of configurations consisting of a taper tapering in the direction of the tip, a shoulder, a corner, a portion outward of said engagement first section external surface, and combinations thereof. If the needle receiver internal surface (21a) does not delimit projections (21a′) that define a circle with an inner diameter short enough to enable the projections to impart the force on the engagement first section (28b) strong to hold the needle inside the needle receiver during retraction of the needle from the ferrule hollow and through the substrate than the ferrule engagement second section (28d) extends into the needle receiver hollow (21b). Further the projections (21a′) impart a force on the ferrule engagement second section (28d) strong enough to hold the needle (28) inside the ferrule (20) during retraction of the needle (28). During retraction of the needle (28), the needle extracts the ferrule (20) from the ferrule hollow (22) and through the substrate (30). The substrate (30) in the substrate capture chamber (24b) formed with said surface of the probe (24). The imparted force can exceed 5 newtons; it can exceed 10 newtons; it can exceed 19 newtons; it can exceed 30 newtons. It can be from 10 to 30 newtons; it can be from 15 to 30 newtons it can be from 15 to 25 newtons. It can be about 20 newtons+−3 newtons. When the projections (21a′) engage the ferrule engagement second section (28d), the needle (28) and ferrule (20) are in a suture commenced state, stage 1b. See FIG. 7B. The inner diameter of the circle formed by the projections (21a′) and unprojected internal surface (21a″) long enough to accommodate the engagement second section (28d).

Also in the ferrule hollow (22) resides a suture retainer (23) of the ferrule (20) having a suture (25) connected thereto. The suture retainer (23) connects to the needle receiver (21). The suture retainer (23) forms a portion of the ferrule (20).

A flexibly resilient holder (36) resides in a holder hollow (38) delimited by surface of said probe (24). A holder retainer (40) connects to the holder. The holder (36) removably resides in the in a retainer hollow (41) delimited by an internal surface of the retainer. The holder retainer resides in the holder hollow (38). The holder retainer (40) removably couples to the probe (24) with a friction fit between the internal probe surface delimiting the holder hollow (38) and the holder retainer external surface (42). The holder hollow (38) can form a recess in which a portion on the retainer resides (40). The portion residing in the recess forms a section (40a) of the holder retainer of increased width measured along a line perpendicular to the retainer longitudinal axis (41a). The increased width is relative to a section of the retainer more proximate the needle receiver than is the section of increased width (40a). The increased width can be a section of increased diameter.

The needle receiver (21) resides over the holder (36). The holder (36) resides in a compressed state. The compressed holder (36) generates a force acting on said needle receiver (21). The force operates to hold the ferrule (20) in place in the ferrule hollow (22). The force applies pressure against the needle receiver (21). The force extends along a vector perpendicular to the longitudinal axis of the needle receiver (21c) and a longitudinal axis of the ferrule hollow.

Now referring to the ferrule's suture retainer (23) in more detail. The suture retainer (23) includes a longitudinal axis (23a). The suture retainer includes a length extending in the direction of the longitudinal axis. The length extends from a first end of the suture retainer to a second end of the retainer. A first end surface resides (23b) at the first end. A second end surface (23c) resides at the second end. A side surface (23d) extends between and from the first end and to the second end. It can extend from and between the first end surface and the second end surface. The side surface extends cylindrically about the retainer's longitudinal axis (23a).

The suture retainer includes a surface delimiting a hollow (23e) extending in the direction and along the length of the suture retainer. The hollow (23e) and delimiting surface are internal to the retainer's side surface (23d). The internal surface is cylindrical about the axis (23a) and together with the hollow (23e) forms a cylindrical chamber. The chamber opens through the first end surface (23b) at a first entry; the first entry opens through the first end surface (23b) and into the hollow. The first entry opens to environment external to the retainer. The hollow (23e) opens through the second end surface (23c) at a second entry which opens through the second end surface (23e) and into the hollow. The second entry opens to environment external to the retainer. The cylindrical chamber (23e), of the suture retainer (23) has a diameter of about 0.006 inches to 0.014 inches and preferably 0.01 inches. The suture size varies in diameter in increments of 0.002 to 0.003 inches. The suture (25) resides fixed to said suture retainer (23). A portion of the suture resides inside said retainer hollow (23e). The suture extends through first entry into the hollow (23e). A portion of said suture (25) resides outside said retainer (23). The suture retainer can be made from plastic and is preferably made from metal. The hollow (23e) and chamber is formed by using a laser. The suture (25) is held in the retainer (23) by crimping the retainer.

Now still referring to the ferrule (20) in more detail, the needle receiver includes a length extending in the direction of the longitudinal axis (21c). The length extends from and between a first end of the needle receiver and a second end of the needle receiver. The first end can be a place along the length of the receiver. A first end surface (21d) resides at the first end. A second end surface (21e) resides at the second end. An external side surface (21f) extends along said length between and from the first end and to the second end. The side surface extends about the receiver's longitudinal axis (21c). The external side surface (21f) includes a plurality of recessed surfaces (22b). Each recessed surface formed from a detent (21f′). There are a plurality of three detents arranged equidistantly about the longitudinal axis. Outside of the surface areas forming the detents resides un-recessed portions (21f″) of the side surface. Un-recessed surface (21f″) resides between each adjacent pair of detents (21f′). Each detent (21f′) resides adjacent with two other detents (21f′). Each detent has a second end (21g) proximate the second end (21e′) and end surface (21e) of the needle receiver and distal the first end (21d′) of the needle receiver. Un-recessed side surface (21f″) resides between said first end of said detent and said second end of said needle receiver.

The needle receiver hollow (21b) and internal surface (21a) extend in a direction and along the length of the needle receiver longitudinal axis (21c). The internal surface (21a) extends about the longitudinal axis (21c). The hollow (21b) and delimiting surface (21a) are internal to the receiver's external side surface (21f). Portions of the internal surface (21a) delimit a plurality of projection (21a′) end surfaces of inward extending projections (21a′). The projection end surfaces reside inward towards the receiver longitudinal axis (21c). They reside inward of other portions (21a″) of said internal surface (21a). The projections (21a′) extend inward towards the needle receiver longitudinal axis (21c). Each projection (21a′) end surface resides opposite a different one of said recesses of a detent (21f). Each projection (21a′) is formed with a different one of said detents (21f′). There are a plurality of three projections (21a′) arranged equidistantly about the longitudinal axis (21c). Each projection (21a′) resides adjacent another two projections (21a′). The projection end surfaces form a periphery of a circle that has a diameter. Outside of the surface areas forming the projections (21a′) resides un-projected portions (21a″) of the internal surface (21a). Un-projected (21a″) surface resides between each adjacent pair of projections (21a′). The un-projected surface (21a″) forms a periphery of a circle with a larger diameter than the circle formed by the projections (21a′). Each projection has a second end (21a′″) proximate the second end (21e′) at the second end surface (21e) of the needle receiver and distal the first end (23d′) at the first end surface (23d) of the needle receiver (21). Un-projected internal surface (21a″) resides between said second end (21a″) of said projections (21a′) and said second end at the second surface (21e) of said needle receiver (21).

The needle receiver's hollow (21b) and internal surface (21a) form a chamber. The receiver chamber opens through the first end surface (21d) at a first entry; the entry opens through the first end surface (21d) and into the hollow (21b). The first entry opens to environment external to the needle receiver (21). The chamber opens through the second end surface (21e) at a second entry which opens through the second end (21e) surface and into the hollow. The second entry can open to environment external to the needle receiver.

The suture retainer (23), fixedly connected to the needle receiver (21), can include a portion external to the receiver (21) and receiver hollow (21b). The portion external extends away from the needle receiver first (21d′) and second end (21e′). A portion of the suture retainer (23) extends through the second entry of said needle receiver chamber at the second end (21e′) and said portion resides in said hollow (21b). The suture (25) extending outside of said suture retainer (23) can reside outside of said needle receiver chamber (21b). Alternatively the suture retainer (23) can reside completely in the needle receiver chamber (21a, 21b). In this confirmation at least a part of the suture (25) would still reside outside the suture retainer (23) and needle receiver chamber (21a, 21b).

In still more detail, the holder (36) includes a longitudinal axis (36a). A holder internal surface (36b) extends about said axis and delimits a hollow (36c). The holder (36) includes a length that extends in the direction and along the length of the holder longitudinal axis (36a). The holder (36) is resiliently compressible along its length along the longitudinal axis (36a). The holder (36) is also resiliently compressible along a length extending in the direction of the holder's transverse axis. The holder (36) is resiliently flexible. The holder (36) resides in a compressed state when the needle receiver (21) resides inside the probe ferrule hollow (22) and over the holder (36). When the ferrule (20) resides completely outside the ferrule hollow (22), the holder (36) resides in the uncompressed state. In the compressed state, the holder (36) displaces a lesser volume and has a shorter length along its longitudinal axis (36a) than when in the uncompressed state. In the compressed state the holder (36) imparts a force on the ferrule (20) and in particular the needle receiver (21). The force operates to hold the needle receiver (21) in place in the ferrule hollow (22). The force applies pressure against the needle receiver (21). In the present example the holder's longitudinal axis (36a) intersects said needle receiver's, ferrule hollow's, tubes, and needles longitudinal axis at an angle of between 45 degrees and 135 degrees. In the present example the holder's longitudinal axis (36a) is substantially normal to these axis.

The holder retainer (40) retains the holder (36) in the holder hollow (38). The holder retainer (40) positions the holder in the holder hollow (38) relative to the ferrule hollow (22), ferrule (20), suture retainer (23), and needle receiver (21). It further positions the holder (36) relative to the needle receiver's, ferrule hollow's, tube's, and needles longitudinal axis. A friction fit between the holder retainer (40) and probe internal surface delimiting said holder hollow (38) holds the holder retainer (40) in position. More particularly the holder retainer includes the projection (40d). The projection (40d) resides proximate the holder retainer first end (40b) and distal holder retainer second end (40c). The projection (40d) forms a friction fit with the probe internal surface delimiting the holder hollow (38) The projection (40d) can comprise a plurality of discrete sections. Each section can be spaced apart from an adjacent section. Each section adjacent two other sections. Between each adjacent section can reside holder retaining external surface (40d′) inward of each adjacent section's end surface. Inward being towards an inner of the holder retainer. The holder (36) connects to the holder retainer. The holder (36) resides in hollow (41) delimited by an internal surface of the holder retainer (40). The holder (36) connects to the holder retainer via a friction fit. The holder (36) retainer prevents the holder (36) from moving out of an orientation during the suturing process. The suturing process includes coupling of the needle (28) to the needle receiver (21), removal of the ferrule (20) coupled to the needle (28) from the ferrule hollow (22) during retraction of the needle tip (28a) towards the probe progression hollow (24c). The holder retainer (40) can be made of plastic and other suitable materials.

In more detail the needle (28) resides in an elongate tube (26a) of the suturing device. The needle (28) includes a first end portion (28e) and a second end portion oppositely oriented from the first end portion. The tip (28a) of the needle resides at the first end portion (28e). The tip (28a) includes an exterior surface that tapers to a point at one end of the tip. The tip extends away from and from the ferrule engagement first section (28b) of the needle (28). The ferrule engagement first section external surface (28b′) extends about the needle's longitudinal axis (28c). It extends circumferentially about the axis. The ferrule engagement first section (28b) includes a first width. The width is a first diameter delimited by the external surface (28b′). The external surface faces a direction normal to the needles longitudinal axis (28c).

The ferrule engagement first section (28b) extends away from and from the ferrule engagement second section (28d) of increased width relative to the width of the ferrule engagement first section (28b). The widths are diameters. The ferrule engagement first section (28b) extends from the ferrule engagement second section (28d) of increased diameter towards the tip (28a). The engagement second section (28d) of increased width is a section of increased diameter relative to the engagement first section (28d). The engagement second section of increased diameter includes external surface facing (28d′) in a direction normal to the longitudinal axis (28c). The engagement second section (28d) of increased diameter can include the end portion (28d″). The end portion can form structure along which the engagement second section (28d) steps up to its place of maximum diameter which is greater than the maximum diameter of the engagement first section (28b). An end of the engagement first section resides proximate said end portion (28d″) of the engagement second section (28d) of increased diameter and distal the needle tip (28a).

The engagement second section (28d) of increased diameter extends away from a section of increased width. (28g). The section of increased width is an increased diameter. The maximum diameter of the section of increased diameter exceeds the maximum diameter of the engagement second section (28d). The engagement second section (28d) of increased diameter extends away from the section of increased diameter (28g) towards the tip. A second end portion (28h) of the engagement second section (28d) opposite the first end portion (28d″) resides proximate an end portion of the section of increased (28g) diameter and distal the needle tip (28a). The second end portion (28h) of the engagement second section (28d) terminates at the end portion of the section of increased diameter. The end portion (28i) of the section of increased diameter (28g) has an external surface which forms a taper section that extends away from the section of increased diameter's (28g) portion of maximum diameter and towards the tip (28a). The end portion (28i) of the section of increased diameter's external surface could have a configuration selected from a group of configurations consisting of a taper tapering in the direction of the tip, a shoulder, a corner, a portion outward of said engagement second section (28d) external surface, and combinations thereof.

The probe (24) in more detail includes a first entry (22c) opening into the ferrule hollow (22) from an external environment to the ferrule hollow (22). The first entry (22c) opens to into an open area (24a) which itself opens into a suture capture chamber (24b). A second entry (22d) opens into the ferrule hollow (22) at an end of the hollow opposite the first entry (22c). The suture (25), connected to the suture retainer (23), extends out from the second entry (22d) when the ferrule (20) resides in a suture ready state.

The probe includes the needle progression hollow (24c). The hollow opens out of the probe to environment external to the progression hollow. It opens out of the hollow and into the probe open area at the first needle exit (24c′). The needle progression hollow (24c) includes an entry (24c″) through which the hollow opens into the suture device tube (26a). An operator, through an actuator, moves the needle progressively out of the progression hollow (24c). During the progressive movement the needle tip (28a) moves towards the needle receiver (21) first end (21d′) and ferrule hollow first entry (22c).

The holder hollow (38) opens into the ferrule hollow (22) when emptied of the holder (36) and holder retainer (40). The holder hollow (38) also opens through the probe at an entry. It opens to environment external to the probe when the holder hollow (38) is emptied.

Still referring to the probe (24) in more detail, the probe includes a stadium configured port (24d) opening into the substrate capture chamber (24b). A flat arcuate surface that resides in a recessed manner delimits the open area (24a). The open area (24a) and substrate capture chamber (24b) employ a probe construction like that shown in U.S. Pat. No. 11,103,236, Suturing System. The tube (26a) extends between the probe (24) and handle (45) of the suture device (26). The tube (26a) interconnects the handle (45) to the probe (24). The handle (45) carries an actuator like the actuator in U.S. Pat. No. 11,103,236. A drive member residing in the tube (26a) and handle (45) interconnects the actuator to the needle (28) at the needle end like that in U.S. Pat. No. 11,103,236. The handle (45) and tube (26a) carry a vacuum line. The line has a vacuum port at one end of the handle. The port connects to a vacuum source of supply. The line includes an entry (24b′) which opens into the substrate capture chamber (24b) to provide a negative pressure. The vacuum line is like the line in U.S. Pat. No. 11,103,236. U.S. Pat. No. 11,103,236 is incorporated herein by reference.

In more detail the ferrule (20), having the suture retainer (23) connecting to the needle receiver (21) and a suture (25) connected the suture retainer (23); the ferrule hollow (22); the holder (36); the holder retainer (40); and the needle (28) take on different orientations relative to each other during the suturing process. In the suture ready state, the ferrule (20) as described above, resides in the ferrule hollow (22). More particularly the needle receiver (21) resides in the recess (22b) of the ferrule hollow (22). The suture (25) resides outside and inside the ferrule hollow (22). The needle receiver at the first end (21d′) opens towards the progression hollow exit (24c′). The needle receiver (21) resides over the holder (36). The holder (36) resides in the holder hollow (38). The holder (36) is compressed. The holder retainer (40) resides in the holder hollow (38) and connects to the holder (36).

In the suture commenced state, stage 1a, the tip (28a) of the needle (28), the ferrule engagement first section (28b) of the needle (28) resides in the needle receiver chamber (21b′). The engagement first section (28b) opposite the inward projections (21a′). The projections (21a′) impart a force on the ferrule engagement first section external surface (28b′) strong enough to hold the needle (28) inside the needle receiver (21) during retraction of the needle tip (28a) towards the progression hollow exit (24c′). The projections (21a′) impart a sufficient force on the engagement first section (28b) to hold the needle (28) in the needle receiver (21) during retraction of the needle from the ferrule hollow (22) and through the substrate (30). The second section (28d) resides outside the suture retainer (23). The ferrule engagement second section (28d) accordingly resides in a position relative to the projections (21a′) wherein the projections (21a′) fail to impart a force on the engagement second section (28d) strong enough to hold the needle (28) inside the needle receiver (21) during retraction of the needle tip (28a) towards the progression hollow exit (24c′). The ferrule (20) is in the hollow. The holder (36) remains compressed by the needle receiver (21). The holder (36) retainer remains connected to the holder retainer (40) and remains disposed in the holder hollow (38). The needle extends through a substrate (30).

In the suture commenced state, stage 1b the tip (28a) of the needle (28), the ferrule engagement first section (28b), and the ferrule engagement second section (28d) of the needle resides in the needle receiver chamber (21b′). The engagement second section (28d) opposite the inward projections (21a′). The projections (21a′) impart a force on the ferrule engagement second section (28d) strong enough to hold the needle inside (28) the needle receiver (21) during retraction of the needle tip (28a) towards the progression hollow exit (24c′). The projections (21a′) impart a sufficient force on the engagement second section (28d) to hold the needle (28) in the needle receiver (21) during retraction of the needle (28) from the ferrule hollow (22) and through the substrate (30). The projections (21a′) fail to impart the sufficient force on the engagement first section (28b) to hold the needle (28) in the needle receiver (21) during retraction of the needle tip (28a) from the ferrule hollow (22) and through the substrate (30) and towards the progression hollow exit (24c′). The ferrule (20) is in the ferrule hollow (22). The holder (36) remains compressed by the needle receiver (21). The holder retainer remains connected to the holder retainer (40) and remains disposed in the holder hollow (38). The needle (28) extends through a substrate (30).

In the suture commenced state, the ferrule (20) resides completely outside the ferrule hollow (22) and connects to the needle (28) as described in suture commenced, stage 1a or suture commenced stage 1b. The holder (36) resides in a decompressed state in the holder hollow (38) and in the ferrule hollow (22). In the suture commenced, state, a portion of the suture (25) resides inside, and a portion of the suture (25) resides outside the substrate (30). The suture (30) remains connected to the suture retainer (23); the suture retainer (23) remains connected to the needle receiver (21). The needle receiver (21) resides connected to the needle (28). The ferrule (20) resides on a side of the substrate (30) opposite the side it resides when connected to the needle (28) and in the ferrule hollow (22). The ferrule (20) on the opposite side remains connected to the needle (28).

When the needle (28) resides in the suture commenced state, stage 1a, the force imparted on the needle engagement first section (28b) markedly inhibits movement of the slider (27) forward. The ferrule engagement second section (28d) first end proportion (28d″) can also markedly inhibit movement by way of an interference fit with the needle receiver (21). When the needle (28) resides in the suture commenced state, stage 1b, the force imparted on the needle engagement second section (28d) markedly inhibits movement of the slider (27) forward. The needle receiver (21) fails to impart a force on the engagement first section (28b) to markedly inhibit movement of the slider (27) forward.

As can be easily understood from the foregoing, the basic concepts of the present invention may be embodied in a variety of ways. The invention involves numerous and varied embodiments and methods for making and using such embodiments.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “needle receiver” should be understood to encompass disclosure of the act of “needle receiving”—whether explicitly discussed or not—and, conversely, were there is effectively the disclosure of the act of retracting” such a disclosure should be understood to encompass the disclosure of a “retractor” and even a “means for retracting”. Such alternative terms for each element or step are to be understood to be explicitly included in the description.

In addition, as to each term used it should be understood that unless its utilization in this application is inconsistent with such interpretation, common dictionary definitions should be understood to be included in the description for each term.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 4.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Thus, the applicant(s) should be understood to reserve the right to claim at least: i) the needle ii) the related methods disclosed and described, iii) similar, equivalent, and even implicit variations of the needle iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application includes or contains reference to United States patents, publications. It is not intended that any United States or publication interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice versa as necessary to define the matter for which protection is sought by this application or by any subsequent application or continuation, division, or continuation in part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

We claim:

1. A needle adapted to insert into a ferrule when said ferrule resides in a ferrule hollow formed with surface of a suturing device probe, said needle comprising:
a first end portion;
a tip at said first end portion of said needle, said sip includes an external surface which tapers to a point;
an engagement first section, said tip extending away form said needle engagement first section, an external surface of said engagement first section about a longitudinal axis of said engagement first section; said external surface delimiting a first diameter;
an engagement second section having a second diameter, said second diameter greater than said first diameter, an external surface of said engagement second section about a longitudinal axis of said engagement second section; said external surface delimiting said second diameter, an external surface delimiting an end portion, said external surface delimiting said end portion delimiting a configuration selected from a group of configurations consisting of a taper tapering in the direction of the tip, a shoulder, a corner, a portion outward of said engagement first section external surface, and combinations thereof, wherein,
said engagement first section extends away from said engagement second section towards said tip; and wherein
when said needle oriented in a suturing commenced state, stage 1a, the tip of the needle and the ferrule engagement first section of the needle resides in a needle receiving hollow delimited by an internal surface of a needle receiver of said ferrule, said internal surface of said needle receiver delimiting said hollow imparts a force on the ferrule engagement first section strong enough to hold the needle in the needle receiver during retraction of the needle from the ferrule hollow formed with surface of the suturing device probe, towards a progression hollow exit formed by surface of said suturing device probe, and through a substrate in a chamber of the suturing device probe, an inner diameter of a circle delimited by said internal surface of said needle receiver too short to accommodate said ferrule engagement second section in said needle receiver hollow; said engagement second section outside said needle receiver; and
when said needle oriented in a suturing commenced state, stage 1b, said internal surface of said needle receiver delimiting said hollow imparts a force on the ferrule engagement second section strong enough to hold the needle in the needle receiver during retraction of the needle from the ferrule hollow of the suturing device probe, towards the progression hollow exit, and through the substrate in the chamber of the suturing device probe, said inner diameter of said circle delimited by said internal surface of said needle receiver too long to enable said internal surface of said needle receiver to impart the force on the engagement first section strong enough to hold the needle in the needle receiver during retraction of the needle from the ferrule hollow of the suturing device probe, towards said progression hollow, and through said substrate in said chamber of the suturing device probe.

2. The needle adapted to insert into the ferrule of claim 1,
wherein said internal surface of said needle receiver which delimits said needle receiving hollow forms a plurality of projections, and
wherein when said needle oriented in said suture commenced state, stage 1a, said projections impart said force on said ferrule engagement first section strong enough to hold the needle in the needle receiver during retraction of the needle from the ferrule hollow formed with surface of the suturing device probe, towards said progression hollow exit formed by surface of said suturing device probe, and through said substrate in the chamber of the suturing device probe and towards the progression hollow exit formed by surface of said suturing device probe.

3. The needle adapted to insert into the ferrule of claim 2,
wherein when said needle oriented in said structure commenced state, stage 1b, said projections impart said force on said ferrule engagement second section strong enough to hold the needle in the needle receiver during retraction of the needle from the ferrule hollow formed with surface of the suturing device probe, towards a progression hollow exit formed by surface of said suturing device probe, and through said substrate in the chamber of the suturing device probe and towards the progression hollow exit formed by surface of said suturing device probe.

4. The needle adapted to insert into the ferrule of claim 3,
wherein when said needle oriented in said suture commenced state, stage 1a, said inner diameter of said circle delimited by said projections, said inner diameter too short to accommodate said ferrule engagement second section in said needle receiver hollow.

5. The needle adapted to insert into the ferrule of claim 4,
wherein when said needle oriented in said suture commenced state, stage 1a, said inner diameter of said circle delimited by said projections, said inner diameter too long to impart the force on the engagement first section.

6. The needle adapted to insert into the ferrule of claim 1,
wherein when said needle oriented in said suture commenced state, stage 1a, a compressible and resilient holder resides compressed by said ferule in a compressed state.

7. The needle adapted to insert into the ferrule of claim 6,
wherein when said needle oriented in said suture commenced state, stage 1b, a compressible and resilient holder resides compressed by said ferule in a compressed state.

8. The needle adapted to insert into the ferrule of claim 7, wherein said holder resides in a holder hollow delimited by surface of said suture device probe.

9. The needle adapted to insert into the ferrule of claim 8, wherein said holder connects to a retainer.

10. The needle adapted to insert into the ferrule of claim 9, wherein a friction fit between an external surface of said holder and an internal surface connects the holder to the retainer.

11. The needle adapted to insert into the ferrule of claim 10, wherein the holder extends into a hollow delimited by said internal surface of said retainer.

12. The needle adapted to insert into the ferrule of claim 11, wherein the holder extends into a hollow delimited by said internal surface of said retainer.

13. The needle adapted to insert into the ferrule of claim 12, wherein the retainer resides in the holder hollow.

14. The needle adapted to insert into the ferrule of claim 13, wherein a projection of said needle receiver delimits a needle receiving hollow and projects inward towards a longitudinal axis of said needle receiver.

15. The needle adapted to insert into the ferrule of claim 14, wherein said suture retainer resides in a hollow delimited by said internal surface of said needle receiver.

16. The needle adapted to insert into the ferrule of claim 15, wherein said hollow in which said suture retainer resides continuous with said needle receiver.

17. The needle adapted to insert into the ferrule of claim 16, wherein said suture retainer includes internal surface delimiting a hollow.

18. The needle adapted to insert into the ferrule of claim 17, wherein said suture extends into said hollow delimited by said internal surface of said suture retainer.

19. The needle adapted to insert into the ferrule of claim 18, wherein said suture retainer comprises metal.

20. The needle adapted to insert into the ferrule of claim 18, wherein each of said plurality of projections is formed with a detent.
